(19) United States
(12) Patent Application Publication
(10) Pub. No.: US 2004/0180343 A1
(43) Pub. Date: Sep. 16, 2004

(54) Compositions and methods for detecting nucleic acids

(76) Inventor: Georg F. Weber, Chestnut Hill, MA (US)

Correspondence Address: FISH & RICHARDSON PC, 225 FRANKLIN ST, BOSTON, MA, 02110, US

(21) Appl. No.: 10/387,305

(22) Filed: Mar. 12, 2003

(51) Int. Cl. C12Q 1/68

(52) U.S. Cl. 435/6

(57) ABSTRACT

The methods and compositions of the invention provide means of detecting nucleic acids. The methods employ hybridization of test nucleic acids to nucleic acid probes or analogs thereof (e.g., peptide nucleic acid probes).

### TECHNICAL FIELD

This invention relates to compositions and methods for detecting nucleic acids.

### BACKGROUND

The ability to analyze nucleic acids has significantly advanced our understanding of cellular function in both healthy and diseased animals. Virtually any gene sequence can be detected, and the sequence of that gene and its level of expression can be determined in virtually any context. More specifically, oncogenes have been studied in the context of cancer; viral genes and those of other pathogens have been studied to detect and monitor infectious disease; and receptors, ion channels, and other cell surface-associated proteins that mediate cellular signals have been studied in the context of numerous disease states.

Traditional methods of detecting nucleic acids require sequence-specific nucleic acid probes. These traditional methods include Southern and Northern blot analyses and methods based on the polymerase chain reaction (PCR). PCR and reverse-transcriptase PCR (RT-PCR) permit analysis even when the starting quantities of nucleic acids are small. For example, PCR-based techniques can be used to analyze nucleic acids expressed in a single cell or “one cell” equivalent (see, e.g., Edmands et al., PCR Methods Applic. 3:3 17-319, 1994; Rodriguez et al., Nucleic Acids Res. 20:3528, 1992). As useful as these techniques have been, they each have shortcomings. For example, the absolute amount of product generated in a PCR reaction does not always bear a consistent relationship to the amount of test sequence present at the initiation of the reaction. Non-linear amplification can make it difficult to perform quantitative analyses, which are often required to assess clinical specimens. In addition, when RNA samples are analyzed by RT-PCR, irregular efficiencies in both reverse transcription and amplification steps are potential sources of variability.

### SUMMARY

The compositions and methods of the present invention provide a new way to detect nucleic acids. According to the methods, a sample suspected of containing at least one test nucleic acid sequence is contacted with two probes, each of which contains a sequence that is at least partially complementary to a distinct portion of the sequence of the test nucleic acid. One of the two probes, possibly by virtue of associated moieties (described further below), serves to immobilize, or anchor, the test nucleic acid to a substrate, and the second of the two probes, also by virtue of associated moieties (also described further below) facilitates detection of the test nucleic acid. If the test nucleic acid is present within the sample, it will hybridize to the first and second probes, forming a complex we refer to herein as a triplex (first probe—test nucleic acid—second probe). The steps that can be carried out to generate the triplex are described below, but we note here that the sample can be exposed to the probes sequentially or simultaneously. The sequence of a probe is “at least partially complementary to” the sequence of the test nucleic acid when the extent of identity between the sequences allows them to hybridize to one another under, for example, physiological conditions or under a condition used to carry out an assay of the invention.

The probes of the invention include nucleotide sequences or analogs thereof (e.g., peptide nucleic acids (PNAs)) and, as noted above, these probes may also include additional moieties (which may also be referred to as binding partners). For ease of reading, we may refer to any probe, regardless of its sequence, content, or association with other agents (e.g., moieties that facilitate detection or immobilization), as simply a “probe”. However, the invention encompasses both “simple” nucleic acid probes or analogs thereof (as well as pairs of nucleic acid probes or analogs thereof that bind distinct regions of a test nucleic acid) and “complex” nucleic acid probes or analogs thereof (i.e., nucleic acid sequences (e.g., oligonucleotides) or analogs thereof (e.g., PNAs) that are associated with (e.g., conjugated to or joined to, whether by covalent or noncovalent bonds) moieties that facilitate immobilization and/or detection of the test nucleic acid. The probe that facilitates labeling is conjugated to an agent that can be detected, either directly or by virtue of a secondary label or reaction (this probe may be referred to as the labeling probe), and the other probe is conjugated, directly or indirectly, to a substrate such as a bead or plate (this probe may be referred to as the anchoring probe). When the anchoring probe is bound, directly or indirectly to a substrate, unbound probe and any other non-complexed material, can be washed away, leaving a triplex bound to the substrate. Detection of the labeled probe, whether it remains in the form applied or as modified following subsequent steps to create a detectable signal, indicates that the test nucleic acid is present in the sample. Further, the methods of the invention can be quantitative; the quantity of the test nucleic acid in a sample can be determined.

More specifically, the invention includes methods of detecting a test nucleic acid in a sample by (i) providing a sample that is suspected of containing a given test nucleic acid sequence, (ii) contacting the sample with a first probe, (iii) contacting the sample with a second probe, and (iv) detecting a triplex that contains the test nucleic acid and the first and second nucleic acid probes. The first and second probes can contain sequences that are complementary to, and therefore hybridize to, distinct sequences of the test nucleic acid. Detecting the triplex indicates that the test nucleic acid is present within the sample (we expect samples will typically contain a number of different nucleic acid molecules).

The test nucleic acid can be any RNA or DNA; the probes described above can be designed to recognize any given nucleic acid sequence (strategies for designing probes are well known in the art, and the characteristics of probes useful in the present invention are described further below).

As noted above, one of the two probes can be bound to a substrate, either directly or by way of a moiety that anchors the probe to the substrate, and the other probe can be conjugated to a detectable agent (i.e., to a fluorophor or other agent that can be detected by virtue of its own properties) or to a molecule that can be recognized by virtue of its ability to participate in a reaction that produces a signal (e.g., a protein that can be recognized by an antibody or an enzyme that produces a detectable reaction product). The anchoring probe can be anchored to any solid or semi-solid substrate, such as a plate, bead, membrane, frit, slide, chip, or the like. To facilitate attachment of the anchoring probe to the substrate, the substrate can be coated, in whole or in part, with any of a number of agents. For example, a substrate can be coated or partially coated with a histidine-binding agent such as a nickel-chelating group (e.g., nitriloacetic acid (NTA) or imidotriacetic acid (IDA)), and the anchoring probe can be coupled to one or more histidine residues or analogs thereof. Similarly, the substrate can be coated with a coating that contains glutathione, and a glutathione-S-transferase (GST) molecule can be attached to the anchoring probe. In another scenario, the substrate coating can include maltose, and a maltose-binding protein (MBP) can be conjugated to the anchoring probe.

More complex attachment schemes are also useful. For example, a “capture” oligonucleotide can be attached to the substrate by any of the same methods used to attach an anchoring probe to a substrate. When a capture oligonticleotide is used, the sequence of the anchoring probe can be extended so that it contains sequence that will hybridize with the test nucleic acid (as described above) and sequence that will hybridize with the capture oligonucleotide. The extension of the anchoring probe may be referred to herein as a “tail,” and it can extend from either the 5′ or the 3′ end of the anchoring probe. When the tail sequence hybridizes to the capture oligonucleotide, the anchoring probe becomes linked to the substrate. Similarly, moieties (or binding partners) that facilitate detection of the test nucleic acid or help to immobilize it can be attached to either the 5′ or the 3′ end of a given probe. More specifically, a moiety can be conjugated on the 7-deaza or C-8 positions of a purine or deazapurine, or to the C-5 position of a pyrimidine.

In yet another embodiment, the substrate's coating can include avidin or streptavidin, and a biotin or biotinylated molecule can be conjugated to the anchoring probe (the converse can also be used—biotin or a biotinylated molecule can be attached to the substrate and the anchoring probe can be attached to avidin or streptavidin). There are still other alternatives. For example, the substrate's coating can include an antibody that recognizes a protein or peptide conjugated to the anchoring probe. The protein or peptide can be essentially any protein or peptide; all that is required is that the protein or peptide be one that is specifically bound by an antibody.

The labeling probe can be detected by virtue of association with a detectable label. For example, the labeling probe can be conjugated to biotin or a biotinylated protein or to a substance such as digoxygenin, flavin adenine dinucleotide (FAD), c-myc, hemagglutinin (HA), or the like. If the labeling probe is conjugated to biotin, it can be detected with avidin or streptavidin, and if the labeling probe is conjugated to a protein or peptide, the binding partner can be detected with an antibody. The labeling probe can also be conjugated to an enzyme such as horseradish peroxidase (HRP), beta-galactosidase, luciferase, or chloramphenicol acetyltransferase (CAT). Alternatively, the labeling probe can be conjugated to a fluorescent molecule such as green fluorescent protein (GFP), blue fluorescent protein (BFP), fluorescein isothiocyanate (FITC), rhodamine, Texas Red, phycoerythrin (PE), or allophycocyanin (APC);

many fluorescent compounds are commercially available (see, e.g., Molecular Probes, Eugene Oreg.) and are suitable for use in the present assays. The detectable label can also be, for example, FAM, TET, HEX, JOE, TAMRA, ROX, an aromatic-substituted xanthene dye, 4,7-dichloro-fluorescein, 4,7-dichloro-rhodamine, a cyanine dye, ³²P, ³³P, or other radioisotope. The triplex can be detected in a variety of ways depending on the nature of the label. For example, the triplex can be detected by measuring absorbance, fluorescence, or radioactivity.

The signal from the labeling probe can be used to quantitate the amount of triplex formed in an assay of the invention, the amount of triplex formation being indicative of the amount of test nucleic acid in the sample.

The sample containing the test nucleic acid can be a cell lysate or other disrupted or fractionated cell preparation; all that is required is that the nucleic acid to which the assay is directed be sufficiently available to the anchoring and labeling probes. The sample can also be a preparation of DNA or RNA, and the RNA can be a poly(A)⁺ RNA. The sample can originate from a cell culture or an animal or human subject, and cells obtained from these sources may have been exposed to a pathogen, such as a virus, bacterium, or mycobacterium or other agent (e.g., a potential or actual therapeutic agent, a toxin, or a metabolite). The probes used to detect the nucleic acid can be designed to detect nucleic acids from the pathogen. Thus, the methods of the invention can be used to determine whether a patient has been infected with a pathogen. Moreover, the cells can be used soon after they are harvested or they can be frozen or otherwise preserved and examined later. While we expect the sample will often contain a nucleic acid of interest, the invention is not so limited. The assays described herein can be used to determine or help confirm that a sample is free of nucleic acids or free of a particular nucleic acid. This information could be useful in clinical tests (e.g., diagnostic tests) or in forensic analysis (e.g., the methods could help establish whether a sample (e.g., a sample of blood or skin) contains DNA from a suspect or victim).

While cells may be used as obtained, they may also be manipulated in some way, either before or after they were obtained from a source. For example, the cells (or the person or animal from which they were obtained) may have been exposed to a therapeutic agent or procedure (e.g., a radiotherapy or imaging procedure). Accordingly, the methods of the invention include those aimed at assessing the impact of a treatment (or potential treatment) on gene expression in a cell. As noted above, the cells may have also been exposed to a toxic substance or metabolite.

Materials that can be used to carry out the methods described herein are also within the scope of the invention. These materials include a probe (e.g., the anchoring probe described above, with or without “tail” sequences, and the labeling probes, whether immediately detectable or not), combinations of probes (e.g. an anchoring probe and a labeling probe that bind distinct sequences on a given test nucleic acid or sets of paired anchoring probes and labeling probes that can be used to detect a given population of test sequences (e.g., a population of cytokines or other growth factors, cancer-associated genes, or genes associated with neurodegenerative diseases (e.g., huntingtin))), or a probe and another agent. For example, the invention features a capture oligonucleotide and a complementary, extended anchoring probe. A capture oligonucleotide and an anchoring probe are complementary when the extended or tail portion of the probe has a sequence sufficiently identical to a portion of the capture oligonucleotide that the two sequences hybridize to one another under a condition used to carry out the methods of the invention. The invention also features solid supports (e.g., beads, plates, or chips) to which an anchoring probe or a set of anchoring probes have been attached. For example, the invention features a chip (e.g., a microchip) or an array (e.g., a microarray) to which anchoring probes have been attached. The probes may hybridize to a set of related or unrelated test sequences, and the substrate can carry a variable number of anchors (from a few (e.g., 3, 4, 5, 6, 8, 10, or 12) to a few dozen (e.g., 12, 24, 36, 48, 60, 72, 84, or 96) to a few hundred (e.g., 200, 300, 400, 500, 600, 700, 800, 900, or 1000) to a few thousand (e.g., 2,000, 3,000, 4,000, 5,000, 8,000, 10,000, 12,000, 15,000, 18,000, 20,000, 22,000, 25,000, 27,000, 30,000, 40,000, 50,000, 60,000, 70,000, 80,000, 90,000 or 100,000 or more), or any number between these. The microchip or microarray can be packaged together with instructions for its use and a key that identifies the positions of the various anchoring probes.

Other materials of the invention can also be packaged as kits. For example, the kits can contain one or more of the probes or other materials or reagents described herein or any combination thereof, together with instructions for use (e.g., printed, audio- or audiovisual instructions). Moreover, the kits can contain a variety of probes useful in detecting a variety of related or unrelated nucleic acids (related nucleic acids can be splice variants or nucleic acids that encode proteins within a given family (e.g., a family of growth factors or signaling molecules, or transcription factors). The nucleic acids can also be related by virtue of expression or misexpression in connection with a particular disease (e.g., cancer, a neurological disease, or an autoimmune disease) or condition. Moreover, as described above, the substrate-bound probes (i.e., the anchoring probes or capture oligonucleotides) can be arrayed on a chip or otherwise ordered to facilitate high throughput screening. Accordingly, arrayed anchoring probes are within the scope of the invention. The kits can also contain materials useful in control experiments (e.g., a nucleic acid sequence that can be used as a positive control for a test nucleic acid). The kits can also contain various buffers (e.g., a hybridization buffer and a wash buffer), blocking agents (i.e., an agent that binds nonspecifically to proteins and surfaces to decrease the non-specific binding of other molecules), and reagents for detecting the triplex (e.g., reagents to carry out assays for CAT or beta-galactosidase).

The materials and methods described herein may have one or more advantages over other nucleic acid assays. For example, the present methods are PCR-independent and, as such, are not subject to amplification errors. Another advantage may be the sensitivity of the methods; the high affinity of PNA to nucleic acids allows for the capture and quantitation of low amounts of nucleic acids. As implied above and described further below, the assay can be configured for high-throughput applications (e.g., for screening assays and gene expression analyses). The invention can provide a kit for detecting a sequence-specific nucleic acid in a sample.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### BRIEF DESCRIPTION OF THE DRAWINGS

FIG. 1 is a schematic diagram of a DNA backbone and a PNA backbone.

FIG. 2 is a graph plotting absorbance versus known concentrations of standard control Bcl-xS RNA detected in accordance with the methods described herein. The line marked with diamonds represents a classic standard curve, illustrating the absorbance produced in the presence of known amounts of control Bcl-xS RNA (X-axis represents amount of standard control RNA). The flat line marked with squares (absorbance is constant and near 0) represents the absorbance produced by known amounts of Bcl-xS antisense RNA (used as a negative control) (X-axis represents amount of standard antisense RNA). The line marked with squares that is descending with increasing amounts of RNA (X-axis)), represents the effect of increasing amounts of antisense RNA on the absorbance of 80 ng standard RNA (X-axis represents amount of antisense RNA present in the test sample).

FIG. 3 is a standard curve plotting absorbance versus increasing concentrations of standard Bcl-xS RNA. The lines illustrate the increase in observed absorbance levels when the test samples were spiked with 0 ng, 500 ng or 1000 ng unlabeled total RNA extracted from resting cells.

FIG. 4 is an ethidium bromide-stained agarose gel revealing semi-quantitative RT-PCR results. The RT-PCR experiments measured Bcl-xS RNA levels in AF3-G7 T cells stimulated for six hours with increasing amounts of anti-CD3 antibody. Actin RNA was used as an RT-PCR control.

FIG. 5 is a graph plotting the amount of total Bcl-xS RNA determined to be present in AF3.G7 T cells following activation with increasing concentrations of anti-CD3 antibody. The amount of Bcl-xS RNA was determined by the assay of the invention described herein.

FIG. 6 is an ethidium bromide-stained agarose gel revealing semi-quantitative RT-PCR results. The RT-PCR experiments measured Bcl-xS RNA levels in AF3-G7 T cells stimulated with 0.5 μg anti-CD3 antibody for increasing amounts of time. Actin RNA was used as a control. “No template” indicates that RNA was not present in the initial reverse transcription reaction.

FIG. 7 is a graph plotting the amount of total Bcl-xS RNA determined to be present in AF3.G7 T cells following activation with 0.5 μg anti-CD3 antibody for increasing amounts of time. The amount of Bcl-xS RNA was determined by the assay of the invention described herein.

### DETAILED DESCRIPTION

The methods described herein can be used to detect and, if desired, quantitate nucleic acids, including all types of DNA and RNA, whether from a human, other animal, or pathogen (such as a virus, bacterium, or mycobacterium). The nucleic acid of interest may be referred to herein as a “test” nucleic acid. The test nucleic acid can be detected in a variety of ways, but regardless of the precise steps carried out, their order, or the precise reagents used, the test nucleic acid is detectable when it forms a triplex that includes itself and two probes. Each of the probes recognizes a distinct portion of the sequence of the test nucleic acid, at least one of the probes can be detected, and at least one of the probes can attach the triplex to a solid support. For example, a sample, which may or may not contain the test nucleic acid, can be contacted with the first probe, and if the test nucleic acid is present in the sample, the text nucleic acid will hydridize to the first probe to form a complex. The first probe can be either a labeling probe or an anchoring probe (the anchoring probe being attached or attachable to a solid support). The sample, which then may or may not contain a complex, is then contacted with the second probe. Once the second probe hybridizes to the complex, the complex may be referred to as a triplex. Following triplex formation, unbound probes, nucleic acids, and other cellular material can be washed away. Once the unbound labeling probe is removed, one can detect the test nucleic acid by detecting the labeling probe. Further, by quantitating the amount of the labeling probe, one can quantitate the amount of test nucleic acid in the sample.

While the steps that can be carried out, and their order, are described further below, we note here that the triplex can be assembled in a variety of ways. In various embodiments, one can: (1) attach the anchoring probe to the substrate, expose the anchored probe to the sample, and expose complexes that form between the anchoring probe and nucleic acids in the sample to the labeling probe; (2) expose the sample to the labeling probe and expose complexes that form between the labeling probe and nucleic acids in the sample to an anchoring probe, which may or may not be anchored to a substrate; or (3) expose the sample to the labeling probe and the anchoring probe, then attach the triplex to a substrate by way of the anchoring probe. An anchoring probe or an array of anchoring probes may be obtained already attached to a substrate. In that event, of course, no “attaching” step would be required. In any of these scenarios, one or both of the probes used can include peptide nucleic acids (PNAs).

There are many circumstances in which one desires to detect or quantitate a nucleic acid. The present assays can be used in any of the same circumstances in which nucleic acids are currently assessed (by, for example, Northern blotting or a polymerase chain reaction assay). For example, the present methods can be used in any research project in which gene expression is in question. For example, the methods of the invention can be used to screen animals (such as mice or sheep) in order to determine whether a transgenic founder has been created or whether a gene targeting experiment (to create, for example, a “knock out” animal) has been successful.

The present methods can also be used to measure splice variants of genes that are difficult to resolve on Northern blots. For example, the anchoring probe can be designed to target a common region of an mRNA transcript, and an array of labeling probes can be designed to identify specific splice variants. For example, using the invention with probes identical to a mouse RNA or DNA sequence to hybridize with a test nucleic acid sample from a human can identify a homologous gene, or a gene with similar structure and/or function in the human.

The assays can also identify and monitor gene expression in a disease process or during the course of a therapeutic regime. For example, the methods can detect an increase in HER-2 RNA levels (i.e., an increase in HER-2 gene expression), which occurs in 30% of human breast cancers (Slamon et al., Science 235:177-182, 1987). The assays can also be used to identify points at which gene expression is regulated. For example, by comparing mRNA and protein levels of a gene in question, it is possible to identify those genes that are being regulated post-transcriptionally.

The methods of the invention can also be used to detect foreign nucleic acids (i.e., nucleic acids not normally expressed in a given tissue or in a given species). For example, the assays of the invention can be used to detect bacterial genes in humans or other animals (e.g., in livestock) or viral nucleic acids (e.g., nucleic acids from human immunodeficiency viruses (HIV), human papilloma viruses, influenza viruses, Epstein-Barr viruses, etc . . . ). For example, the methods can be used to detect and/or monitor the levels of an HIV gag, pol, or env sequence (or any other HIV sequence) in a patient through the disease progression to AIDS (Furtado et al., J. Virol. 69:2092-2100, 1995). It has been suggested that higher levels of circulating HIV and failure to effectively control virus replication after infection may be associated with a negative disease prognosis; in other words, there may be an association between virus level (HIV replication) and the pathogenesis of the disease (Paitak et al., Science 259:1749-1754, 1993). Accordingly, an accurate determination of HIV nucleic acid levels early in an infection may serve as a useful tool in diagnosing illness, while the ability to correctly monitor the changing levels of viral nucleic acid in one patient throughout the course of an illness may provide clinicians with critical information regarding the effectiveness of treatment and progression of disease.

Nucleic acid probes, analogs thereof, and moities to which they may be attached. In the paragraphs that follow, we describe probes (simple and complex) that can be used in the methods of the invention. These probes can include conventional nucleic acids (e.g., polymers of naturally occurring nucleotides) as well as nucleic acid analogs (e.g., polynucleotides having chemical groups or linkages different from those found in nature or in conventional nucleic acids; the analogs can also be PNAs). For example, the probes of the invention can include nucleotides linked by phosphorothioate linkages (e.g., phosphorothioate oligodeoxynucleotides or S-oligonucleotides), mixed phosphorothioate and phosphodiester linkages (e.g., S—O-oligodeoxynucleotides or phosphodiester/phosphorothioate 2′-O-methyl-oligoribonucleotides; Zhou et al., Bioorg. Med. Chem. Lett. 8:3269-74, 1998), methylphosphonate-phosphodiester modifications (e.g., MP-O-oligonucleotides; Zhao et al., Antisense Res. Dev. 3:53-66, 1993), or morpholino oligonucleotides (e.g., Schmajuk et al., J. Biol. Chem. 274:21783-89, 1999).

The labeling and anchoring probes contain sequences that are complementary to non-overlapping or substantially non-overlapping sequences present in the test nucleic acid (i.e., a DNA or RNA in a sample). The sequences of the probes should be sufficiently complementary to the sequence of a test nucleic acid that the probe and the test nucleic acid will hybridize to one another under the conditions in which the assay is carried out. These conditions are described further below. Here, we note that, as in any hybridization procedure, the temperature and concentration (e.g., salt concentration) of the solutions used are such that the probe and test nucleic acid bind to one another, but the probe does not significantly bind to non-test nucleic acids. That is, the conditions permit selective or specific binding. The binding affinities of the two probes need not be identical, however. The probes can be designed such that one binds to the test nucleic acid under more stringent conditions than the other. When the test nucleic acid is exposed to the probes sequentially, it may be that the second hybridization event is carried out under conditions less stringent than the first.

Similarly, the two types of probes need not be equally specific; one probe may recognize more test nucleic acids than another. For example, where the test nucleic acid is a particular spliced mRNA isoform, the sequence of the anchoring probe can be complementary to an exon common to multiple isoforms of the target mRNA. Following exposure of the sample to the anchoring probe, a variety of nucleic acids (i.e., any of those containing the common exon) may hybridize with the anchoring probe. The labeling probe, however, can be designed to hybridize with a unique exon or a sequence spanning an exon-exon junction that is unique to the particular mRNA isoform of interest; if the complexes created by the first hybridization event are exposed to such a labeling probe, the signal generated from the detection assay will be specific for the particular isoform of interest, despite the lack of specificity of the anchoring probe. Similarly, to detect a splicing intermediate, the labeling probe can recognize a specific exon-intron junction.

In another scenario, the anchoring probe can be designed to hybridize with a highly conserved sequence, such as a sequence encoding a DNA-binding domain or an enzyme signature motif, such as a kinase or ATPase domain. The labeling probe can then be designed to recognize a sequence that is less well conserved (and more unique to the test nucleic acid). The signal generated from the detection assay will be specific for the nucleic acids that contain the less well-conserved sequence. In addition to facilitating detection of low quantities of test nucleic acid, strategies that increase specificity from one hybridization event to the next can also reduce “background” noise generated by non-specific signals (more standard, blot-based methods of detecting nucleic acids, including Southern and Northern blotting, can be difficult to interpret due to high background signaling).

The specificities of the anchoring and labeling probes can be reversed; in the preceding paragraphs we described assays in which the specificity of the labeling probe was greater than that of the anchoring probe (the anchoring probe hybridizing to more species of test nucleic acids than the labeling probe), however, the specificity of the anchoring probe can be greater than that of the anchoring probe (the labeling probe hybridizing to more species of test nucleic acids than the labeling probe). In the later scenario, the sample can be exposed to the labeling probe in solution and the complexes that form can then be exposed to the anchoring probe; only those that also bind the anchoring probe will be retained on the substrate to which the anchoring probe is bound (or to which it subsequently binds). Nucleic acids in the sample that hybridize with the labeling probe but not the anchoring probe can be washed away and therefore not detected.

The labeling probe and the anchoring probe (or portions thereof) will each bind a portion of the sequence of the test nucleic acid. For example, the labeling probe can be designed to hybridize with the 17 nucleotides at the most 5′ end of a test nucleic acid and the anchoring probe can be designed to hybridize with the 17 nucleotides at the most 3′ end of the same test nucleic acid. Of course, either probe may bind more or less than 17 nucleotides, and the nucleotides bound need not be those at the extreme ends of the test nucleic acid. For example, if a test nucleic acid is 100 nucleotides long, the anchoring probe can bind (e.g., hybridize with) nucleotides 40-60 and the labeling probe can bind (e.g., hybridize with) nucleotides 61-74.

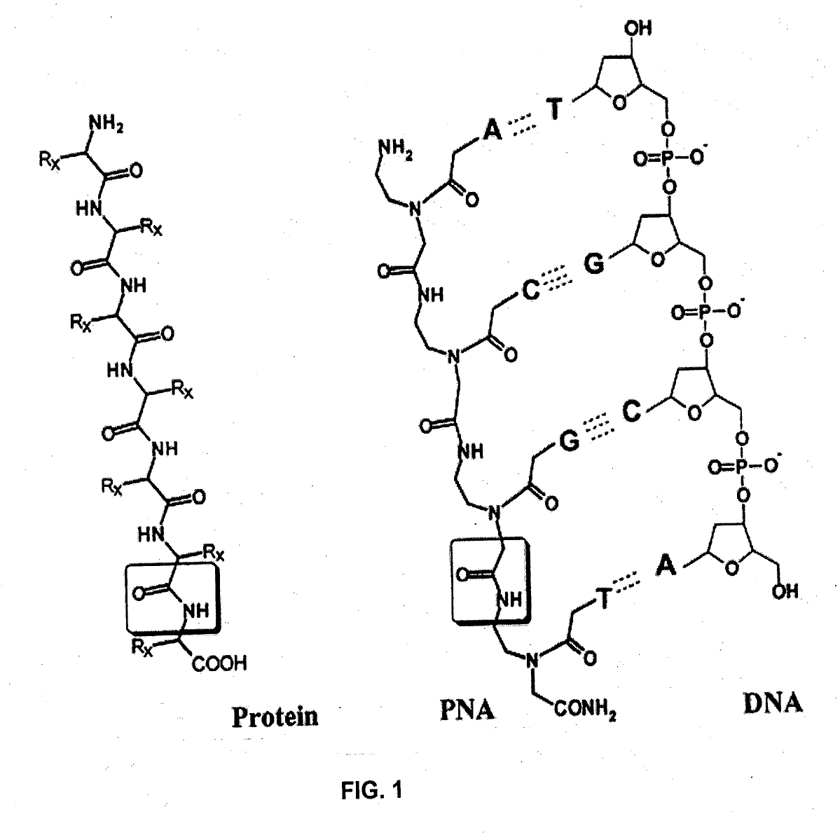

As noted above, the probes of the invention (whether an anchoring probe or labeling probe) can also be PNAs, which encompass nucleic acid analogs in which the backbone is a pseudopeptide rather than a sugar (see FIG. 1). The same is true of capture oligonucleotides; they may be conventional nucleotide polymers, nucleotides with non-naturally occurring chemical groups or bonds, or PNAs. The pseudopeptide (polyamide) backbone typically includes N-(2-aminoethyl)glycine moieties linked by amino bonds. One can attach the bases by, for example, methylene carbonyl linkages (see, e.g., Nielsen, Pharmacol. Toxicol. 86:3-7, 2000; Soomets et al, Front. Biosci. 4:D782-86, 1999 and; Tyler et al., Proc. Natl. Acad. Sci. USA 96:7053-58, 1999). PNAs mimic the behavior of DNA in that they can bind strands of nucleotides having complementary or sufficiently complementary sequences. The neutrality of the PNA backbone can result in stronger bonds between the PNA and a natural gene sequence or oligonucleotide (DNA or RNA; e.g., stronger binding than would be seen between a DNA or RNA probe and a natural gene sequence or oligonucleotide). PNAs are also naturally resistant to nuclease degradation, and their neutral backbones minimize binding to cellular proteins and consequent nonspecific background effects. Derivatives of PNAs (as described, for example, in U.S. Pub. 2002/0187473) can also be used in the methods described herein.

The probes of the invention and the capture oligonucleotides can be synthesized by any method known in the art (e.g., they can be synthesized, either in solution or on a solid phase). The PNA synthesis method can generally be carried out in a manner similar to the oligonucleotide synthesis methods conventionally known in the art (see, for example, Neilson et al., Acc. Chem. Res., 24:278, 1991). Any of the probes used in the methods of the invention can vary in length (e.g., they can be about 10, 12, 15, 17, or 20 bases long). More important than the length of the probe per se is its ability to specifically hybridize with a test nucleic acid under the conditions in which the assays of the invention are carried out; the probe can be of any length so long as it specifically binds the test nucleic acid.

At some point in the detection process, the anchoring probe is attached to a substrate such as a plate or a bead (alternative times for attachment are described further herein). The precise character of the substrate (e.g., its size, shape, and material content) can vary so long as it provides the means for immobilizing a triplex and allows one to subsequently detect the labeling probe present within the triplex. For example, the substrate to which the anchoring probe is attached can be formulated from polystyrene, controlled-pore-glass, silica gel, silica, polyacrylamide, magnetic beads, polyacrylate, hydroxyethylmethacrylate, polyamide, polyethylene, polyethyleneoxy, or copolymers or grafts of any of the above solid substrates. The configuration or format of the solid substrate can be, for example, small particles or beads of approximately 1 to 50 microns in diameter (e.g., 5-10, 10-25, 25-30, 30-40, or 40-50 microns in diameter), membranes, frits, slides, plates, micromachined chips, alkanethiol-gold layers, non-porous surfaces, or polynucleotide-immobilizing media.

As discussed above, the substrate can be (but is not necessarily) coated with a material to facilitate attachment of the anchoring probe. The substrate can be coated, for example, with a nickel-chelating agent such as nitriloacetic acid (NTA) or imidotriacetic acid (IDA), a capture oligonucleotide, avidin or streptavidin, or an antibody. The nature of the coating can vary so long as it attaches the anchoring probe, either directly or indirectly, to the substrate. For example, a molecule within the coating can bind the probe or a binding partner attached to the probe. For example, the probe can be attached to a histidine residue or multiple histidine residues (such as in a 6-His tag; in this event the 6-His tag is the binding partner) and the substrate can be coated with a nickel-chelating agent such as nitriloacetic acid (NTA) or imidotriacetic acid (IDA). Alternatively, the probe can have a FLAG-tag (Hopp, et al., Bio/Technology 6:1204-1210, 1988) attached at one end, and anti-FLAG antibodies can be attached to the substrate or the substrate can be coated with FlAsH (Fluorescein Arsenical Helix binder) resin (Thorn et al., Protein Sci 9:213-217, 2000) for capture. Other binding partners that can be conjugated to an anchoring probe include glutathione-S-transferase (GST) and maltose-binding protein (MBP), which will bind substrates coated with glutathione and maltose, respectively.

Alternatively, a “tail” comprising nucleic acids or analogs thereof can be made to extend from the 5′ or 3′ end of an anchoring probe, with the sequence of the tail being sufficiently complementary to the sequence of a capture oligonucleotide that hybridization between the tail of the probe and the capture oligonucleotide anchors the anchoring probe to the substrate. The capture oligonucleotide can be attached to the substrate in a variety of ways, including any of the methods described herein for attaching the anchoring probe to the substrate. For example, the surface of the substrate can be coated with avidin or streptavidin to anchor a biotin-labeled or biotinylated capture oligonucleotide to a substrate.

Biotin, and any of the other binding partners useful in the methods of the invention, can be attached to either the 5′ or the 3′ end of the probe, and attachment (to the probe, whether an anchoring probe or labeling probe (discussed further below) or to a capture oligonucleotide) can be facilitated through a spacer (e.g., an 8-atom spacer (Biotin-ON) or a 15-atom spacer (Biotin-TEG), or a nucleotide such as thymidine (Biotin-dT)).

As noted above, the anchoring probe can also be immobilized by way of an antibody. Thus, the anchoring probe can also include, in addition to the nucleotide sequence that hybridizes with the test nucleic acid (i.e., in addition to the “simple” probe), a peptide, protein, or any entity that can be bound by an antibody (binding partners such as these may be referred to herein as epitopes). The anchoring probe can be conjugated to, for example, digoxygenin, fluorescein, flavin adenine dinucleotide (FAD), c-myc, hemagglutinin (HA), or any other such epitope. The epitope can then be recognized by (and specifically bound by) an antibody attached to the substrate.

The moieties described above can be arranged differently to achieve the same result (i.e., attachment of the anchoring probe to a substrate). For example, we described above a substrate coated with a nickel-chelating agent, avidin or streptavidin, or an antibody. In that event, the anchoring probe would be conjugated to, respectively, a histidine-containing tag, biotin (or a biotinylated protein), or a protein or peptide specifically bound by the antibody. These moieties can also be configured differently; the anchoring probe can be conjugated to the nickel-chelating agent, avidin or streptavidin, or an antibody, and the substrate can be coated with a coating containing, respectively, a histidine-containing tag, biotin (or a biotinylated protein), or a protein or peptide that is specifically bound by the antibody. This flexibility extends to any pair of binding partners. For example, the anchoring probe can be conjugated to glutathione-S-transferase (GST) or a maltose-binding protein (MBP), and the substrate's coating can include glutathione or maltose, respectively. Alternatively, the anchoring probe can be conjugated to glutathione or maltose, and the substrate's coating can include, respectively, GST or MBP.

The labeling probes can also be conjugated to moieties that allow, or facilitate, detection. Moreover, some of the moieties associated with the labeling probe can be the same as those associated with the anchoring probe. For example, the labeling probe can be conjugated to biotin, avidin, streptavidin, or to an antibody. While these moieties can be used to facilitate detection or to immobilize the triplex, it is preferable not to use the same moieties in a given assay to achieve both aims; preferably, the moieties (or binding partners) on the labeling and anchoring probes used in a given assay are distinct. For example, if the anchoring probe is conjugated to biotin, it is preferable to attach the labeling probe to a binding partner other than biotin (and vice versa).

The procedures used to detect binding partners (e.g., biotin and avidin) are known in the art and can be used in the context of the present assay. For example, if biotin is conjugated to the labeling probe, then avidin-peroxidase or streptavidin-peroxidase can be used to enzymatically produce a chromophore, which can be subsequently detected by measuring absorbance. Avidin or streptavidin (or other binding moieties) can be attached to a variety of other enzymes that act as reporter compounds; these other enzymes include, but are not limited to, alkaline phosphatase, β-galactosidase, luciferase, chloramphenicol acetyltransferase (CAT), and the like. In yet another detection scheme, avidin, streptavidin, or another binding partner can be coupled to a fluorescent label such as FITC, TRITC, DTAF, Texas-Red, phycoerythrin, allophycocyanin, green fluorescent protein (GFP), blue fluorescent protein (BFP), rhodamine, or any such label (many fluorescent labels are commercially available, and methods for fluorescently labeling a protein or nucleic acid are known in the art).

The labeling probe can, alternatively, include an epitope that is specifically bound, and thereby detected, by an antibody. This antibody (which may be referred to as a “primary” antibody) may be fluorescently tagged or otherwise detectable, or it (the primary antibody) may be bound by a secondary antibody that is fluorescently tagged or otherwise detectable. The epitope can be, or be within, virtually any protein (whether naturally occurring or not), and it can include those described above as useful in immobilizing the anchoring probe (e.g., digoxygenin, fluorescein, FAD, c-myc, HA, or any other such epitope).

To detect a labeling probe conjugated to an epitope (and consequently detect a test nucleic acid hybridized to the labeling probe), one can contact the epitope with an antibody coupled to a fluorescent tag (as noted above) or coupled to any of a wide variety of enzymes. Suitable enzymes include, but are not limited to, horseradish peroxidase (HRP), alkaline phosphatase, β-galactosidase, luciferase, CAT, and the like. Methods for generating a reaction product with these enzymes and detecting that product are known in the art and can be carried out in the context of the present assays. Methods are also known to increase the sensitivity of a given detection method. For example, a binding partner such as avidin can be linked to polymerized HRRP (Sigma Chemical Co., St. Louis, Mo.), which typically couples around ten molecules of enzyme to each molecule of avidin (unpolymerized HRP couples only a single enzyme molecule to each molecule of avidin). Alternatively, the antibody can be labeled with a fluorescent label such as FITC, TRITC, DTAF, Texas-Red, phycoerythrin, allophycocyanin, GFP, BFP, rhodamine, or any such label.

We have made reference herein to probes (e.g., labeling probes) that can be detected (e.g., visualized) “directly.” This can be done by, for example, incorporating a fluorescent molecule into the nucleic acid analog probe itself. Exemplary fluorescent molecules include FAM, TET, HEX, JOE, TAMRA, ROX, aromatic-substituted xanthene dyes, 4,7-dichloro-fluoresceins, 4,7-dichloro-rhodamines, and cyanines. The label can also be a radioactive element, such as a ³²P, ³³P, ³H, or ³⁵S isotope incorporated into the structure of the probe. Radiolabels such as these can be attached to any given probe using kinase assays known in the art.

Triplex formation. Any given test nucleic acid can be detected upon formation of a triplex structure: anchoring probe—test nucleic acid sequence—labeling probe. The steps required to assemble the complex can be carried out in various orders (i.e., there is more than one way to create the triplex). The test sequence can be exposed to the anchoring probe and then the labeling probe; to the labeling probe and then the anchoring probe; or to both probes simultaneously or in an overlapping temporal sequence. Moreover, the anchoring probe can be attached to a substrate or it can be free in solution (i.e., not immobilized) when it is exposed to a sample suspected of containing the test nucleic acid. While the compositions and methods of the invention are not limited to those formed by, or that require, any particular biochemical mechanism(s) of action, the three primary components of the triplex are expected to associate by hybridization; by base pairing between nucleotides. Accordingly, the triplex is assembled under conditions in which nucleic acids can hybridize to one another. Conventional methods based on nucleic acid hybridization (e.g., in situ hybridization or genomic or cDNA library screening) often include washing steps designed to reduce non-specific binding, and the methods of the invention can include such steps as well. For example, where one begins with an anchoring probe bound to a substrate and subsequently exposes that probe to a sample containing nucleic acids (under conditions and for a time sufficient to allow hybridization between the anchoring probe and the test nucleic acid), a wash step can be performed before the labeling probe is applied to wash away nucleic acids that have not bound the anchoring probe. Another washing step can be carried out after the labeling probe has been allowed to hybridize with the anchoring probe-test nucleic acid complex. By washing away unbound or non-specifically bound sequences the washing step(s) decrease background noise and help optimize the accuracy of the assay).

Any sample that contains, or is suspected of containing, nucleic acids can be tested according to the methods described herein. For example, the sample can include a cell, or a cell lysate or fraction, obtained from a cell culture (e.g., a bacterial, insect, or mammalian cell culture), a plant, or an animal. The sample can contain nucleic acids from a human subject, whether that subject is presently living or deceased. More specifically, the sample can derive from a tissue or fluid sample, such as blood. In addition to cell lysates or fractions, the sample can include nucleic acids that are largely isolated from other cellular material. DNA, RNA, and poly(A)¹ RNA can be prepared using methods known in the art. If necessary to improve the availability of sequences for hybridization, the sample can be exposed to conditions in which nucleic acids are denatured. For example, the sample can be heated before it is mixed with a probe. For example, the sample can be heated to 50° C., 55° C., 60° C., 65° C., 70° C., 75° C., 85° C., 95° C. or higher, for 3 minutes, 5 minutes, 10 minutes, 15 minutes, or longer.

The sample can be exposed to the first probe (e.g., the anchoring probe) in the presence of a hybridization buffer. The content of the buffer can vary; typically, hybridization buffers are chosen based on the length of the probe, its sequence, and other characteristics. Where the probes and the test nucleic acids have highly complementary sequences (i.e., few, if any mismatches) the hybridization buffer can contain a higher concentration of salt and/or detergent. Where the probes and the test nucleic acids have highly complementary sequences, hybridization can occur under conditions considered high stringency. Conversely, where probes and test nucleic acids have less than identical sequences, or where one wishes to detect a variety of test nucleic acids (e.g., test nucleic acids that contain the sequence of the probe and similar sequences), the hybridization buffer can have a lower concentration of salt and/or detergent; hybridization can be carried out under conditions considered low stringency.

The hybridization buffers can include a neutral solution, such as a phosphate buffered saline (PBS) solution that facilitates hybridization of a particular test sequence with each of the probes (of course, where the probes are applied sequentially, a different hybridization buffer can be used for each application). Variations in the composition of the buffer, including salt and urea concentration; temperature; and hybridization time can be adjusted to optimize hybridization. To minimize non-specific nucleic acid interactions, the concentration of salt, such as NaCl, can be 100-400 mM (e.g., 200-300 mM). Urea concentrations can range from 0.01-1.0 M (particularly for PNA-nucleic acid hybridizations), and the hybridization temperature can range from about 37-80° C. (e.g., 37°, 42°, 50°, 65°, or 75° C. The hybridization reaction can proceed for at least one minute, 5 minutes, 20 minutes, one hour or longer (e.g., 2, 4, 5, 6, 8, 10, 12, or 24 hours).

As noted above, hybridization can be followed by a wash step. In addition to reducing the amount of unbound nucleic acids in the assay, the wash step can wash away unwanted buffer components, such as salt or urea. Where the anchoring probe and the test nucleic acid are hybridized in solution, any subsequent washing steps can be facilitated by attaching the anchoring probe to the substrate.

Any of the solutions used in the course of assembling the triplex can be prewarmed to the temperature at which they will be used. For example, the wash buffer can be prewarmed to the same temperature, or a higher temperature, as that used for the hybridization step. Moreover, washing steps can be repeated; the wash solution can be applied and removed one, two, three, or more times between or following hybridization.

Where the sample containing a test nucleic acid is exposed to the probes sequentially, following hybridization with the first probe and an optional wash, the second probe is brought into contact with the complex (a duplex) formed between the first probe and the test nucleic acid sequence. The second probe is allowed to hybridize to any test nucleic acid bound by the first probe. If there is no test nucleic acid in the test sample, no duplex will form following the first hybridization and wash steps, and consequently, no label will be detected at the conclusion of the assay. For purposes of describing the methods of the invention, we assume that a duplex has formed.

Hybridization of the second probe (the labeling probe, in this example) to the anchored test nucleic acid, completes the formation of the triplex. The second hybridization buffer and hybridization conditions can be optimized as they were for the first hybridization. Preferably, the components and conditions of the second hybridization will not disrupt (or significantly disrupt) binding between the anchoring probe to the substrate.

After hybridization of the second probe (which completes triplex formation), unhybridized labeling probe is washed away from the triplex. This wash can be repeated 2, 3, 4, 5, 6, or more times with fresh wash buffer. The wash buffer can contain a low concentration of detergent and blocking agent.

Using wash buffers can improve the assays in ways that are independent of physically removing non-specifically bound sequences. For example, the components of the hybridization or wash buffers can be altered to minimize any adverse effect on components that may be used in the assay, such as enzymes that may be used in the process of detecting a triplex. For example, the wash buffer can help prevent the inactivation of horseradish peroxidase (HRP) by urea in the hybridization buffer. In addition, a blocking agent can be used to reduce nonspecific binding of the detection agent (the labeled antibody or binding partner (avidin, for example) that binds the moiety on the labeling probe to facilitate detection) to the substrate. Exemplary detergents include Tween-20, Tween-80, SDS, Laureth 12, and NP-40. Detergent concentrations can range from 5-0.001%, depending on the type of detergent used. For example, the wash buffer can contain 0.1-0.01% Tween-20 (e.g., 0.05% 0.06%, 0.07% or 0.08%). Exemplary blocking agents include BSA, polyvinylpyrrolidone (PVP), polyG, polyl, polyC, polyU and tRNA, and the concentrations of these agents and agents like them can range from 0.01% to 5% or more. Use of BSA as a blocking agent, for example, works well in the present assays at a concentration of about 3%.

Detection and quantitation of a test nucleic acid. As described above, significant amounts of unbound probe and nucleic acid from a test sample can be washed away from the substrate before assaying for the detectable label present on, or associated with, the labeling probe. Detecting the labeling probe, which is bound to the test nucleic acid, is equivalent to detecting the test nucleic acid. The label can be detected conveniently while the triplex is attached to a substrate. For example, where the substrate is a plate, the label can be detected using a plate reader. However, if desired, the triplex as a whole or the labeling probe alone can be detached from the substrate before a detection step is carried out. For example, if the triplex is bound by a histidine-Ni-NTA interaction, the substrate can be washed with an imidazole-containing solution to disrupt the histidine-Ni-NTA bonds. The eluted nucleic acid or probe can then be assayed in solution for the detectable label.

The precise method used to detect a label will obviously vary, depending on the nature of the label (e.g., the detection methods will vary depending on whether the label produces a fluorescent signal, a colorimetric change, emits a radioactive signal, etc . . . ). Where the probe is not conjugated to a label that can be detected directly (e.g., a fluorophore), a reaction can be carried out to reveal the labeling probe. For example, a labeling probe conjugated to an epitope or epitope-containing protein can be incubated with a solution containing the appropriate cognate antibody (this, the primary antibody, can be detectable (by virtue of, for example, a fluorescent tag), or the signal can be generated by a detectable secondary antibody). Similarly, where the labeling probe is conjugated to another moiety or binding partner (e.g., biotin), the labeling probe can be detected by exposing the probe to a solution containing the cognate binding partner (e.g., where the probe is associated with biotin, it can be detected following a reaction containing avidin or streptavidin). The cognate binding partner can be conjugated to a fluorescent molecule or an enzyme, just as the labeling probe can be.

As noted above, an enzyme can be used to facilitate detection of the triplex, and the enzyme can be bound directly to the labeling probe or bound to a binding partner that becomes associated with the labeling probe. For example, the labeling probe can include a sequence that hybridizes with a test nucleic acid; that sequence can be joined to biotin; and the biotin can bind avidin or streptavidin that is joined to an enzyme. The enzyme facilitates detection of the triplex following exposure to its substrate. Where the result of the enzymatic reaction is a chromophore, it can be detected, for example, by measuring absorbance with a spectrophotometer.

Plates, including 96-well plates, can be used for the assays described herein and, in that case, a positive signal can be detected using a microplate reader. Beads, including magnetic beads, can also be used. Signals generated by bead-bound triplexes can be detected by, for example, measuring absorbance on a spectrophotometer or measuring fluorescence on a fluorometer. A radioactively labeled triplex can be measured with a scintillation counter or by autoradiography. If the triplex is eluted from the substrate prior to detection, the unattached sample can be detected using similar instruments (e.g., a spectrophotometer where the signal generates a color change or a fluorometer where the signal is fluorescence).

If desired, the signal can be quantitated by measuring absorbance, fluorescence, luminescence, radioactivity, and the like. The quantity of the detectable signal correlates with the quantity of test nucleic acid sequence. To determine the correlation, a standard curve can be constructed. For example, a standard curve can be generated with a sample of purified target RNA at a known concentration (the test sequence can be generated by in vitro transcription). The detectable signal is typically determined for a range of concentrations, such as 1-500 ng or 0-100 ng, in 10, 20, or 25 ng increments. To help control for background signals, the highest amount of control RNA used in construction of the standard curve can be assayed (1) in the absence of the first nucleic acid probe (e.g., the anchoring probe), and (2) in the absence of the second nucleic acid probe (e.g., the labeling probe). Control experiments such as these can reveal the background level of signal, which can then be subtracted from the signal in the test samples.

Those of ordinary skill in the art can perform other, or alternative, control reactions, and these reactions can vary, depending on the type of nucleic acid being assayed. For example, when targeting an SNP, three or more hybridization reactions can be performed. For example, hybridized target nucleic acid samples can be probed with (1) an SNP-specific probe in one sample, (2) a “wildtype” non-SNP-containing probe in a first control sample and (3) a “common” probe in a second control sample. The “common” probe can recognize a region other than that containing the SNP. As all copies of the test nucleic acid should be recognized by the second control test, the quantity of the test nucleic acid can be determined by adding the nucleic acid targets recognized by the SNP-positive probe and those recognized by the “wildtype” control probe.

Compositions and Kits. Many of the material compositions used to carry out the assays described herein are within the scope of the present invention. Moreover, the novel materials of the invention can be packaged, either alone or with existing materials (such as buffers, reagents (e.g., enzyme substrates) and solid supports (e.g., small particles or beads, membranes, frits, slides, plates, micromachined chips, alkanethiol-gold layers, non-porous surfaces, or polynucleotide-immobilizing media)) and sold in the form of a kit.

The invention features a solid support to which an anchoring probe has been (reversibly or essentially irreversibly) bound or to which it can be subsequently bound by way of interaction with a binding partner. For example, the invention features a small particle or bead, a membrane, frit, slide, plate, chip, gold layer, non-porous surface or other medium that has been at least partially coated with a solution that contains one member of a binding pair (e.g., an antibody, epitope, epitope-containing moiety (e.g., a protein), biotin, avidin, streptavidin (or commercially available versions or improvements thereof (e.g., Strep-tag® and Strep-tactin®)) or a capture oligonucleotide, to which an anchoring probe can be attached. For example, the invention features a support to which an epitope is bound; such a support can be used with an anchoring probe that contains a sequence complementary to that of a given test sequence and an antibody that specifically binds the support-bound epitope. In another example, the support is coated, at least in part, with a nickel chelating agent; such a support can be used with an anchoring probe that includes a sequence that binds the target nucleic acid and a histidine residue or histidine tag.

Thus, the invention features a support to which a binding partner has been bound; kits that include an uncoated support and a formulation containing a binding partner (e.g., a solution or resuspendable powder (of, for example, a nickel-chelating agent)); a support to which a binding partner or capture oligonucleotide and an anchoring probe have been bound; and kits that include an uncoated support and/or a formulation containing a binding partner and/or an anchoring probe. Also within the scope of the invention are anchoring probes containing either (1) a sequence that specifically binds the test sequence and a binding partner that facilitates attachment to a support or substrate or (2) a sequence that specifically binds the test sequence and a sequence that specifically binds a capture oligonucleotide.

As noted above, the support structures (which may also be referred to herein as platforms or substrates) can bear more than one anchoring probe. Moreover, these anchoring probes can be placed in positionally distinct locations. For example, the invention features arrays (e.g., microarrays or microchips) on which two or more (e.g., a series) of anchoring probes are positionally displayed. Typically, each well of a 96-well plate can be configured to detect a different test nucleic acid or to serve as a control (e.g., some wells can be for experimental use and others can be used to generate a standard curve for quantitating nucleic acids).

Similarly, the invention features labeling probes that include a sequence that specifically binds the test sequence and a detectable agent (e.g., a fluorophore, chromophore, or radioisotope) or an agent that can be subsequently detected (e.g., an enzyme that, when exposed to a substrate gives rise to a detectable product or an epitope that can be detected when bound by a labeled antibody (or when the primary antibody is bound by a labeled secondary antibody). The labeling probes of the invention can also include a sequence that specifically binds the test sequence and a sequence that specifically binds an oligonucleotide that carries, or that is attached to, a detectable label. Where a fluorophore is used, it can be any of those known in the art or any of those noted above, including FAM, TET, HEX, JOE, TAMRA, ROX, aromatic-substituted xanthene dyes, 4,7-dichloro-fluoresceins, 4,7-dichloro-rhodamines, and cyanines.

In any of the embodiments of the invention, the labeling probe, the anchoring probe, and/or the capture oligonucleotide can include peptide nucleic acids.

Cognate probes can be packaged and sold together. For example, cognate anchoring probes and labeling probes can be packaged and sold together; similarly, cognate capture oligonucleotides and anchoring probes can be packaged and sold together. For example, an anchoring probe containing a sequence that specifically hybridizes with a first portion of a given test nucleic acid, and a labeling probe containing a sequence that specifically hybridizes with a second, distinct portion of the same test nucleic acid can be provided in the same package with, optionally, instructions for use. Any sequence known to exist can be a test sequence. Those of ordinary skill in the art are well able to find the sequence of any gene or nucleic acid sequence of interest. They can, for example, consult databases such as that maintained by the National Center for Biotechnology Information (which is associated with the U.S. National Library of Medicine, 8600 Rockville Pike, Bethesda, Md. 20894).

Without limiting the invention, the following genes are provided as examples of the nucleic acids that can be detected: spp1, β-actin, myosin, Bax-α, Bcl, COX-1, COX-2, CXCR4, eotaxin/CLL11, Fas/TNFRSF6, Fas ligand/TNFSF6, GAPDH, GM-CSF, ICAM-1 (CD54) IFN-γ, IL1β/IL-1F2, IL-2, IL-6, IL-8/CXCL8, IL-10, MCP-1/CCL2, MIP-1α/CCL3, NGF, iNOS, p21, p53, RANTES/CCL5, SDF-1/CXCL12, E-selectin, L-selectin, P-selectin, Survivin, TGF-β1, TNF-α, TRAIL/TNFSF10, VCAM-1, and VEGF. Processed or unprocessed or alternatively processed transcripts, including alternatively spliced isoforms, can be detected. Moreover, target nucleic acids may be those of any species (whether a vertebrate or invertebrate, plant or animal, bacterium, virus, or other pathogen).

In addition to one or more of the components described above, the kits of the invention can also include one or more of the reagents used to detect the label or one or more solutions used in the hybridization and/or washing steps that are carried out in the course of forming the triplex.

The kits can also include reagents, including nucleic acids that can be used in control assays and materials to facilitate conjugation of an anchoring moiety onto the user's anchoring probe. For example, materials in the kit can provide the binding moiety (for example, a histidine or biotin) and compositions to facilitate attachment of the moiety onto the experimental anchoring probe.

The kit can also include a secondary labeling molecule, such as a labeled antibody, to recognize an epitope associated with the labeling probe. The antibody can be conjugated, for example, to any of the fluorescent or chemiluminescent moieties described above. The kit can also include reagents (an enzyme substrate, for example) and instructions for detecting the triplex structure, and, further, instructions for analyzing data generated by the methods of the kit. The instructions can be in any format, including in a printed manual or brochure, audiotape, videotape, or CD-ROM or other computer-readable medium.

In addition to any of the components listed above, a kit can contain materials for in vitro transcription of the test nucleic acid of interest, such as an expression vector that has a multiple cloning site adjacent to a promoter, such as a T7 promoter. The kit can also include a polymerase to facilitate transcription, such as a T7 polymerase. Test nucleic acids generated in vitro by these methods can be used to construct a standard curve and in connection with other control paradigms.

Each component of the kit can be enclosed within an individual container, and all of the various containers can be enclosed within a single package. Compositions and reagents of the kit can be packed lyophilized or as solutions.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Quantitation of Bcl-xS mRNA from Stimulated AF3.G7 T Cells

To demonstrate the utility of the invention, AFR.G7 T cells were stimulated with CD3 antibody, and the methods of the invention were used to identify and quantitate a specific mRNA isoform of the BCL-x gene, Bcl-xS.

Methods. To provide an RNAse free environment, buffer stock solutions were autoclaved, and the working solutions were prepared in sterile containers immediately before use. Pipet tips and tubes were RNAse-free. For the detection of BCL-xS, the 15-mer antisense PNA probe 5′-GCTGTATCCTTTCTG-(HIS)₆-3′ (SEQ ID NO:1) was synthesized such that a 6-His tag was fused to the C-terminal end (PNA-His). This probe recognizes a common sequence of BCL-x mRNA transcripts. The wells of a 96-well nickel-chelate plate (Pierce) were rehydrated in binding buffer (PBS containing 0.1% Triton X-100, 300 mM NaCl and 0.1 M urea). The buffer was then replaced with 500 nM of the PNA-His probe in 100 μl binding buffer, and the plate was incubated at room temperature for one hour. Following two washes with 200 μl of binding buffer, total RNA from AF3.G7 T cells, and RNA for the standard curve were added at 100 μl each and incubated for one hour at 55° C. The wells were washed four times with 200 μl prewarmed (55° C.) binding buffer before addition of 100 μl of the biotinylated antisense PNA probe biotin-OO-GTGTCCTGCTCAAAGC (500 nM in binding buffer). The biotinylated probe was unique for the short form of BCL-xS, as it spanned the splice junction. This hybridization was again facilitated by incubation at 55° C. for one hour. The wells were washed at room temperature five times in wash buffer (PBS containing 0.05% Tween-20 and 3% BSA). The wash buffer served two primary purposes: (1) it prevented the inactivation of the horseradish peroxidase by urea, and (2) it acted as a blocking reagent to prevent non-specific binding of avidin to the wells. After incubation with 100 μl of 2.5 μg/mL avidin-peroxidase in wash buffer for 30 minutes at room temperature and six washes with 200 μl wash buffer, 100 μl detection reagent (e.g., ABTS plus hydrogen peroxide) was pipetted into the wells and the absorbance at 405 nm was recorded after 5, 15, and 60 minutes.

Relevant controls included a standard curve with purified BCL-xS RNA generated from a plasmid by in vitro transcription (0-100 ng). Other controls consisted of a sample well without RNA (to assess PNA-PNA hybridization) and a sample well without PNA-His or biotin-PNA each at the highest amount of BCL-xS RNA from the standard curve (to assess background).

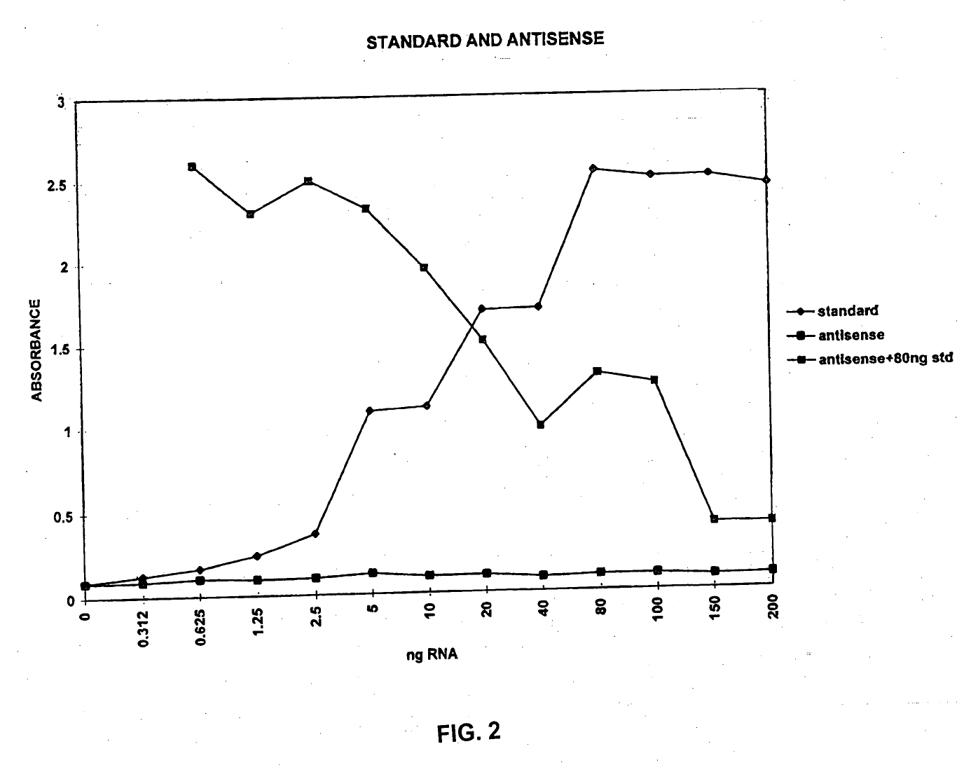

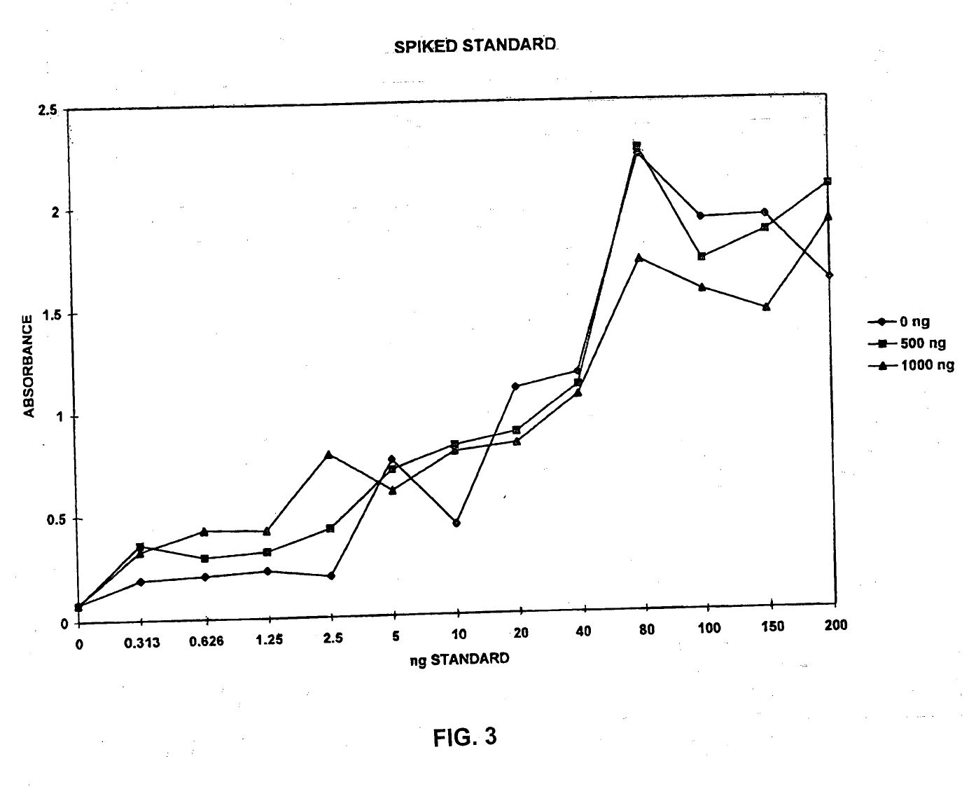

Results. A standard curve was generated from the absorbance profile of purified Bcl-xS RNA (FIG. 2, “standard”). Sense and antisense transcripts were produced by in vitro transcription from a plasmid. FIG. 2 shows that input of an unlabeled antisense transcript (“antisense” in FIG. 2) did not lead to increased absorbance, and the antisense transcript quantitatively competed out 80 ng of standard RNA (“antisense of 80 ng std” in FIG. 2). Additionally, the absorbance profile of standard RNA was not affected by spiking with 500 or 1000 ng of total RNA extracted from resting cells (FIG. 3).

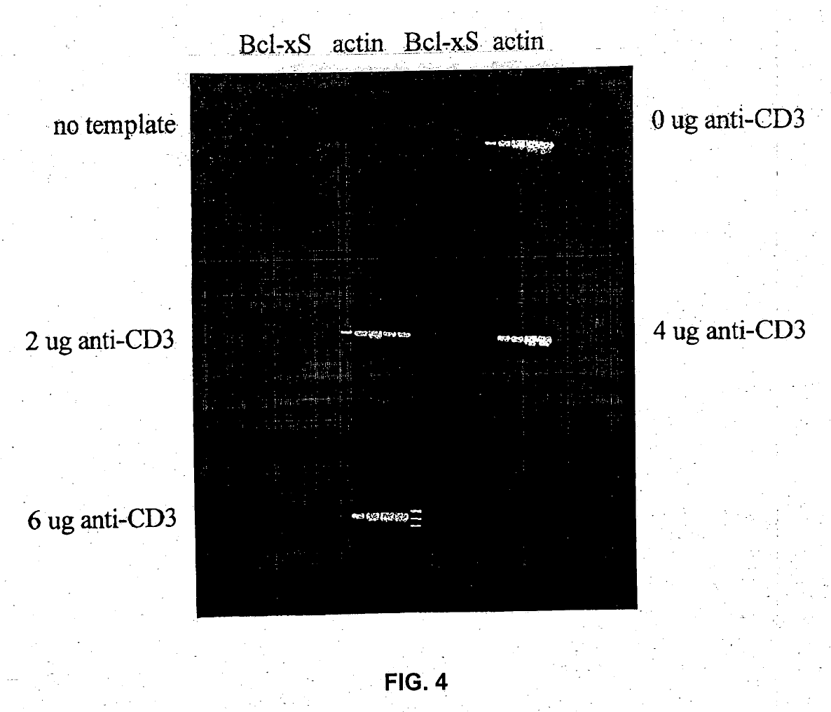

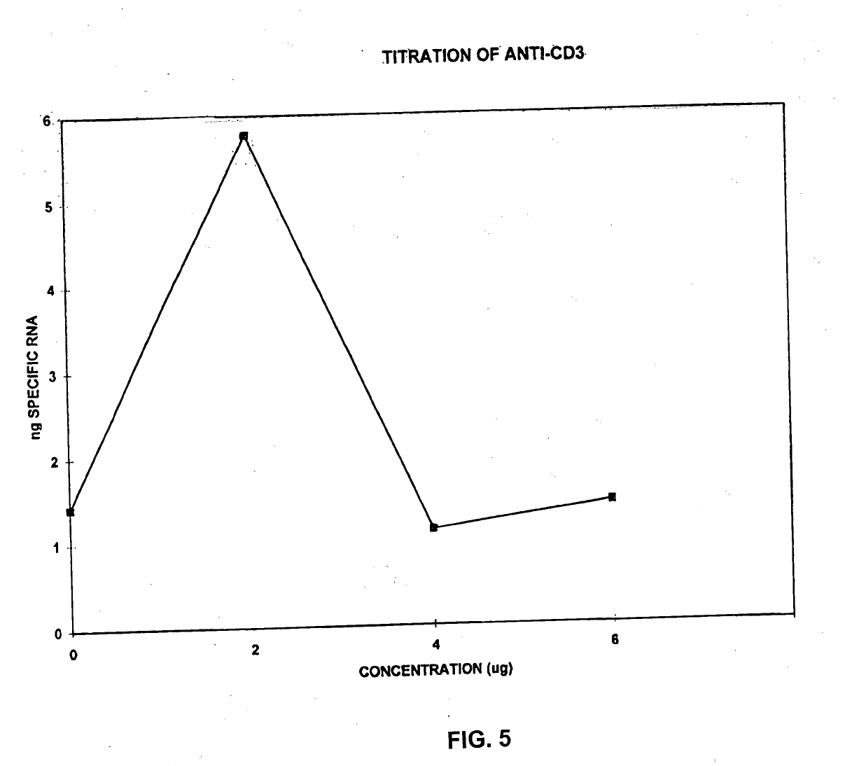

A sample of AF3.G7 T cells was stimulated with increasing amounts of plate-bound anti-CD3 antibody for 6 hours. Total RNA was extracted from the cells and Bcl-xS RNA was assayed either by semi-quantitative RT-PCR with increasing numbers of cycles (in comparison to actin RNA) or by the method described above. FIG. 4 illustrates the semi-quantitative RT-PCR results measuring Bcl-xS RNA at increasing concentrations of anti-CD3. Actin RNA served as a control. FIG. 5 is a graph illustrating the quantity of Bcl-xS RNA at increasing concentrations of anti-CD3 antibody as determined by the assay of the invention.

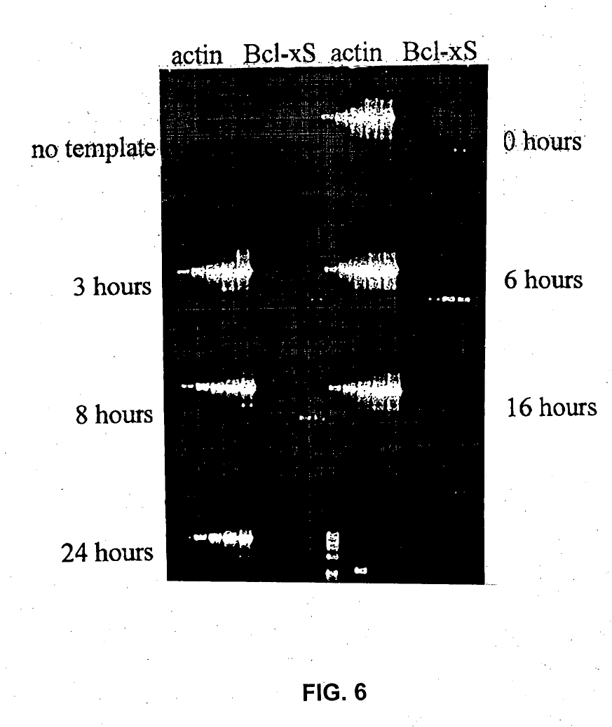

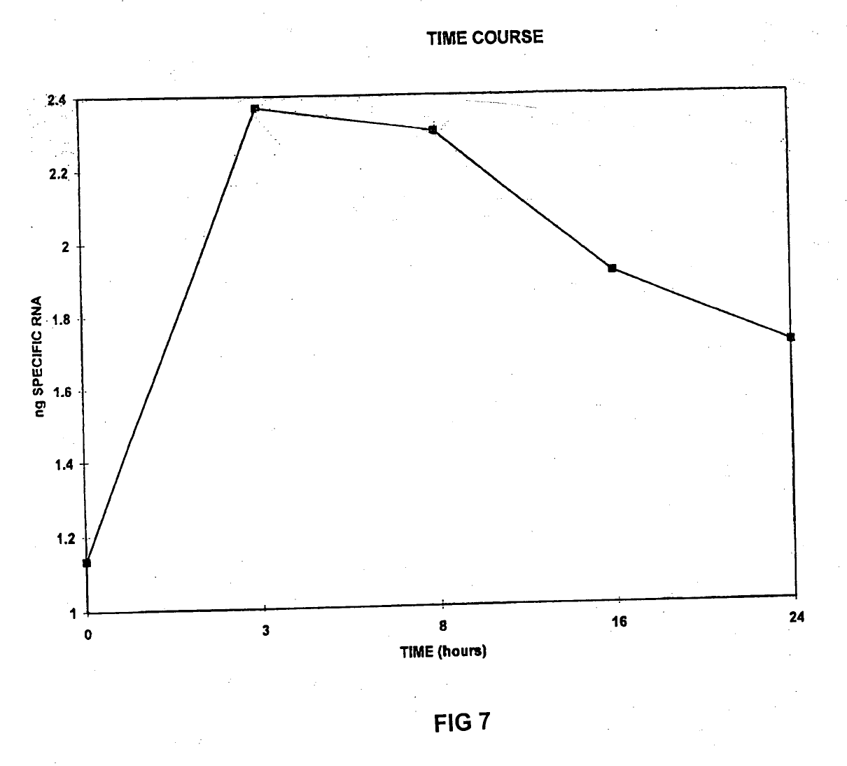

FIG. 6 illustrates semi-quantitative RT-PCR results measuring Bcl-xS RNA at time intervals following stimulation of AF3.G7 T cells after stimulation with 0.5 μg plated anti-CD3 antibody. Actin RNA was used as a control. FIG. 7 is a graph illustrating the quantity of Bcl-xS RNA as determined by the assay of the invention.

What is claimed is:

1. A method of detecting a test nucleic acid in a sample, the method comprising:
(a) providing a sample suspected of containing the test nucleic acid;
(b) contacting the sample with (i) an anchoring probe comprising a peptide nucleic acid (PNA) that specifically binds a portion of the sequence of the test nucleic acid and (ii) a labeling probe comprising a PNA that specifically binds a portion of the sequence of the test nucleic acid that is distinct from the portion bound by the anchoring probe; and
(c) detecting the formation of a triplex comprising the anchoring probe, the test nucleic acid, and the labeling probe, wherein the formation of the triplex indicates that the sample contains the test nucleic acid.

2. The method of claim 1, wherein the sample comprises RNA.

3. The method of claim 1, wherein the sample comprises DNA.

4. The method of claim 1, wherein (a) the contacting step is carried out in solution and the triplex, if formed, is subsequently bound, via the anchoring probe, to a substrate or (b) the anchoring probe is anchored to a substrate prior to carrying out the contacting step.

5. The method of claim 4, wherein the substrate is a plate, bead, membrane, frit, slide, or chip.

6. The method of claim 4, wherein the substrate is at least partially coated with a coating comprising a nickel-chelating group and the anchoring probe further comprises at least one histidine residue.

7. The method of claim 6, wherein the nickel-chelating group is nitriloacetic acid (NTA) or imidotriacetic acid (IDA).

8. The method of claim 4, wherein the substrate is at least partially coated with a coating comprising glutathione and the anchoring probe further comprises at least one glutathione-S-transferase (GST) molecule.

9. The method of claim 4, wherein the substrate is at least partially coated with a coating comprising maltose and the anchoring probe further comprises at least one maltose-binding protein (MBP).

10. The method of claim 4, wherein the substrate is at least partially coated with a coating comprising avidin or streptavidin and the anchoring probe further comprises biotin or a biotinylated protein.

11. The method of claim 4, wherein the substrate is at least partially coated with a coating comprising an antibody and the anchoring probe further comprises a peptide or protein to which the antibody specifically binds.

12. The method of claim 11, wherein the protein is digoxygenin, fluorescein, flavin adenine dinucleotide (FAD), c-myc, or hemagglutinin (HA), or a peptide fragment of any of these proteins.

13. The method of claim 4, wherein a capture oligonucleotide is attached to the substrate and the anchoring probe further comprises a nucleic acid sequence, or an analog thereof, that binds a portion of the sequence of the capture oligonucleotide.

14. The method of claim 13, wherein the capture oligonucleotide is attached to the substrate in one of the following ways: (a) by direct interaction between the oligonucleotide and the substrate or (b) by interaction between a pair of cognate binding partners, one of which is joined to the substrate and one of which is joined to the capture oligonucleotide, wherein the cognate binding partners are (i) a nickel-chelating agent and one or more histidine residues, (ii) glutathione and a GST molecule, (iii) maltose and a maltose-binding protein, (iv) avidin or streptavidin and biotin or a biotinylated protein, or (v) an antibody and a peptide or protein to which the antibody specifically binds.

15. The method of claim 1, wherein the labeling probe further comprises a fluorophor, a chromophor, or a radioactive tag.

16. The method of claim 15, wherein the fluorophor or chromophor is FITC, TRITC, DTAF, Texas-Red, phycoerythrin, allophycocyanin, a green fluorescent protein (GFP), a blue fluorescent protein (BFP), rhodamine, FAM, TET, HEX, JOE, TAMRA, ROX, an aromatic-substituted xanthene dye, 4,7-dichloro-fluorescein, 4,7-dichloro-rhodamine, or a cyanine dye.

17. The method of claim 1, wherein the labeling probe further comprises an enzyme.

18. The method of claim 17, wherein the enzyme is horseradish peroxidase (HRP), alkaline phosphatase, beta-galactosidase, luciferase, or chloramphenicol acetyltransferase (CAT).

19. The method of claim 1, wherein the labeling probe further comprises either (a) avidin or streptavidin, (b) biotin or a biotinylated protein, (c) an antibody or (d) a peptide or protein to which an antibody specifically binds.

20. The method of claim 19, wherein the labeling probe further comprises avidin or streptavidin and the detecting step is carried out by exposing the labeling probe to biotin or a biotinylated protein or peptide conjugated to a fluorophor, chromophor, or radioactive tag.

21. The method of claim 19, wherein the labeling probe further comprises biotin or a biotinylated protein and the detecting step is carried out by exposing the labeling probe to avidin or streptavidin conjugated to a fluorophor, chromophor, or radioactive tag.

22. The method of claim 19, wherein the labeling probe further comprises an antibody and the detecting step is carried out by exposing the labeling probe to a detectably labeled protein or peptide to which the antibody binds.

23. The method of claim 19, wherein the labeling probe further comprises a protein or peptide and the detecting step is carried out by exposing the labeling probe to (a) a detectably labeled antibody that specifically binds the protein or peptide of the labeling probe or (b) an unlabeled primary antibody that specifically binds the protein or peptide of the labeling probe and the detecting step further comprises exposing the unlabeled primary antibody to a detectably labeled secondary antibody.

24. A platform for detecting a test nucleic acid, the platform comprising a substrate and an anchoring probe, wherein the anchoring probe comprises a PNA sequence that hybridizes to the sequence of the test nucleic acid, or a portion thereof, and a moiety that attaches the anchoring probe to the substrate.

25. The platform of claim 24, wherein the substrate is a plate, bead, membrane, frit, slide, or chip.

26. The platform of claim 24, wherein
(a) the anchoring probe further comprises a nickel-chelating agent and the moiety that attaches the anchoring probe to the substrate is one or more histidine residues;
(b) the anchoring probe further comprises one or more histidine residues and the moiety that attaches the anchoring probe to the substrate is a nickel-chelating agent;
(c) the anchoring probe further comprises glutathione and the moiety that attaches the anchoring probe to the substrate is a GST molecule;
(d) the anchoring probe further comprises a GST molecule and the moiety that attaches the anchoring probe to the substrate is glutathione;
(e) the anchoring probe further comprises maltose and the moiety that attaches the anchoring probe to the substrate is a maltose-binding protein
(f) the anchoring probe further comprises a maltose-binding protein and the moiety that attaches the anchoring probe to the substrate is maltose;
(g) the anchoring probe further comprises avidin or streptavidin and the moiety that attaches the anchoring probe to the substrate is biotin or a biotinylated protein;
(h) the anchoring probe further comprises biotin or a biotinylated protein and the moiety that attaches the anchoring probe to the substrate is avidin or streptavidin;
(i) the anchoring probe further comprises an antibody and the moiety that attaches the anchoring probe to the substrate is a peptide or protein to which the antibody specifically binds; or
(j) the anchoring probe further comprises a peptide or protein and the moiety that attaches the anchoring probe to the substrate is an antibody that specifically binds the protein or peptide.

27. The platform of claim 24, the platform comprising at least two distinct anchoring probes, each of which is associated with a spatially distinct location on the substrate.

28. The platform of claim 27, the platform comprising 2-10, 10-25, 25-50, 50-100, 100-200, 200-500, 500-1,000, 1,000-5,000, 5,000-10,000 or 10,000-25,000 distinct anchoring probes, each of which is associated with a spatially distinct location on the substrate.

29. A kit comprising the platform of claim 24 and instructions for its use.

30. The kit of claim 29, further comprising a labeling probe, wherein the labeling probe comprises a PNA that specifically binds a portion of the sequence of the test nucleic acid that is distinct from the portion bound by the anchoring probe.

31. The kit of claim 29, further comprising a hybridization buffer.

32. The kit of claim 31, further comprising a wash buffer.

33. The kit of claim 31, further comprising a blocking agent.

34. The kit of claim 33, wherein the blocking agent is BSA, polyvinylpyrrolidone (PVP), polycG, polyl, polyC, polyU, or tRNA.

35. The kit of claim 30, further comprising a reagent for detecting of the triplex structure.
